(19) United States
(12) Patent Application Publication
(10) Pub. No.: US 2008/0269803 A1
(43) Pub. Date: Oct. 30, 2008

(54) ARTERIOTOMY STAPLE WITH PRIMARY AND SECONDARY PRONGS

(75) Inventor: Ghaleb Sater, Acton, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/739,797

(22) Filed: Apr. 25, 2007

(51) Int. Cl. A61B 17/064

(52) U.S. Cl. 606/219

(57) ABSTRACT

An arteriotomy staple has tissue-engaging prongs arranged in groups each group comprising at least one primary prong and at leas one secondary prong. The secondary prongs are arranged to define a greater transverse span than the primary prongs to provide a back-up for engaging tissue about the arteriotomy in the event that the primary prongs do not work.

### FIELD OF THE INVENTION

The invention relates to devices for closing a percutaneous puncture in a blood vessel after an intravascular procedure and, particularly, to a staple for closing an arteriotomy.

### BACKGROUND

Various cardiovascular procedures, such as angioplasty, stent placement, artherectomy, among others, are performed by inserting into and manipulating within the vasculature, wires and catheters adapted to perform those procedures. Access to the vasculature typically is through the femoral artery and is percutaneous, involving insertion of a needle in the region of the groin to form a track and to puncture and create an arteriotomy in the femoral artery. A guidewire then is advanced through the needle and into the femoral artery. The needle then is removed. An introducer sheath is then advanced over the guidewire. The wire and sheath provide access into the femoral artery, through the arteriotomy, for catheters or other instrumentalities in order to perform the selected procedure.

After the procedure has been completed, the procedural devices are removed and the arteriotomy must be closed. A number of techniques are known to facilitate closure and healing of the arteriotomy. These include, among others, application of pressure at the puncture site for a relatively extended length of time or the use of biological adhesives or plugs adapted to seal the arteriotomy. Also among the techniques for closing the arteriotomy is the use of a staple system such as described in U.S. Pat. Nos. 6,506,210, 6,767,356 and 7,074,232 to Kanner et al., of which the disclosures of the devices and methods are hereby incorporated by reference. The Kanner patents describe a system by which the original introducer sheath is removed, leaving the guidewire in place. Then, an assembly that includes a closure sheath and dilator is advanced along the indwelling guidewire to bring the distal end of the sheath into proximity to the arteriotomy. The closure sheath also carries, at its distal end, an arrangement of wire-like stabilizers that, together with the dilator, pass through the arteriotomy into the artery. The system enables the portions of the stabilizer wires disposed within the artery to be formed into a temporarily enlarged shape that prevents removal of the wires through the arteriotomy and holds the sheath in place. The stabilizers and distal end of the sheath are drawn together to grip the tissue about the arteriotomy and thereby secure and fix the position of the distal end of the sheath over and in alignment with the arteriotomy. The dilator and guidewire then can be removed through the sheath, leaving the closure sheath in place adjacent the outer surface of the artery with the stabilizers within the artery, in place in readiness to provide direct access to the arteriotomy through the sheath.

A catheter-like stapling device, with a staple carried in its distal end, then is advanced through the closure sheath to locate the staple in proximity to the arteriotomy. As described more fully in the Kanner patents, the stapler and sheath include mechanisms by which the staple, when advanced through the sheath, will be oriented in registry with and at a fixed distance from the arteriotomy. When the stapler is actuated, the prongs of the staple first expand and advance toward and into the arterial wall on opposite sides of the arteriotomy. Continued operation of the stapling mechanism draws the prongs of the staple together to draw the edges of the arteriotomy together into approximation and then releases the staple. The stabilizers return to a low profile shape enabling their withdrawal from the artery. With the staple deployed and having closed the arteriotomy the stapling mechanism and sheath may be removed, leaving the staple in place.

Although the guidance and placement system described in the Kanner patents is effective to position the stapler and staple, it would be desirable to have an additional measure of safety in the event that one or more of the prongs of the staple do not engage the tissue as fully as might be desired. Therefore, it would be desirable to provide an arteriotomy staple that provides such an additional measure of safety.

### SUMMARY OF THE INVENTION

In accordance with the invention, the staple is configured to have proximal and distal ends and a longitudinally extending central axis with the proximal end defining a crown. A plurality of prongs extends distally from the crown and terminate in sharp tips adapted to pierce tissue. The prongs are arranged in groups, each of which includes at least one primary prong and at least one secondary prong. The secondary prongs are arranged so that their tips are disposed more radially outward from the central axis of the staple than are the sharp tips of the primary prongs. The secondary prongs may be shorter than the primary prongs. When the staple is advanced distally into tissue about the arteriotomy, the secondary prong or prongs in a group may engage the tissue in the event that their associated primary prongs make insufficient contact with the tissue.

### DESCRIPTION OF THE DRAWINGS

The invention will be appreciated more fully from the following further description with reference to the accompanying drawings in which:

FIG. 1 is a diagrammatic illustration of a prior art arteriotomy staple showing, diagrammatically, the manner in which one of the prongs has misengaged the region of the arteriotomy.

FIG. 2 is an illustration similar to FIG. 1 but showing the manner in which the present invention engages tissue about the arteriotomy in the event of prong misengagement;

FIG. 3 is a plan view of a two-prong staple as may be formed from a flat sheet to include the staple elements of the present invention;

FIG. 4 is a sectional illustration of a staple formed from the blank of FIG. 3 as seen along the plan 4-4 of FIG. 3;

FIG. 5 is an illustration of a blank from which a four-prong staple may be made in accordance with the invention;

FIG. 6 is a somewhat diagrammatic illustration of the distal end of a stapler that may be used to deploy the staple; and

FIG. 7 is an end view of a staple illustrating its detachable connection to a stapler.

### DESCRIPTION OF ILLUSTRATIVE EMBODIMENTS

FIG. 1 shows, diagrammatically, the manner in which one or more prongs 12 of an arteriotomy staple 10 may misalign with the artery 14 or surrounding tissue in the region of the arteriotomy 16. As shown, one of the prongs 12A has missed the intended target tissue 18 adjacent the arteriotomy and has projected into the arteriotomy. Consequently, when this staple is closed, the misaligned prong 12A will not be effective in cooperating with other prongs 12 of the staple to close the arteriotomy.

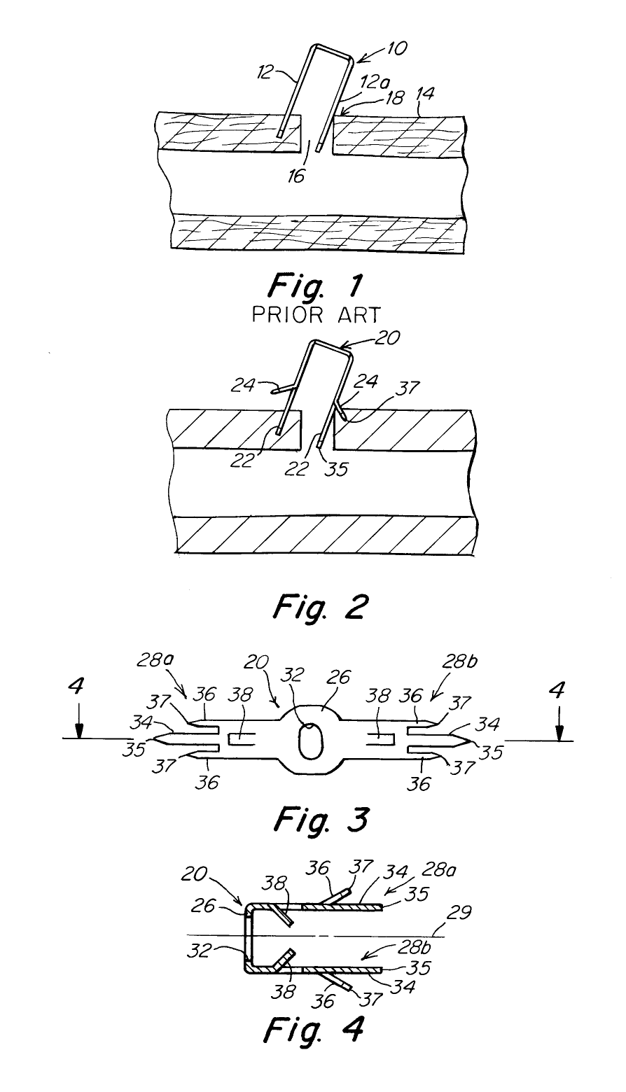

In accordance with the invention, illustrated in similar diagrammatic form in FIG. 2, a staple 20 is provided to have at least two groups of prongs including primary prongs 22 as well as one or more secondary prongs 24 associated with each of the primary prongs. In the event that a primary prong 22 misses or otherwise does not engage satisfactorily with its intended target tissue, the secondary prong 24 associated with that primary prong serves as a back-up to engage the tissue.

One embodiment of the invention is illustrates in FIGS. 3 and 4. FIG. 3 shows a staple blank formed from a flat sheet of material, such as stainless steel or other appropriate biocompatible alloy, or other material that may be formed into the staple 20. The blank is formed to include a crown region 26 and, for a two-prong embodiment (FIG. 3), a pair of prong arrays or groups 28a, 28b. The groups 28a, 28b preferably are arranged diametrically opposite each other. When the blank is formed into a staple by bending the prongs (FIGS. 4 and 6), the prong groups 28a, 28b will have been deformed to extend in a distal direction, generally parallel to the longitudinal central axis 29 of the staple. The crown 30, formed from crown region 26 of the blank, is provided with suitable means, such as a noncircular aperture 32, by which the crown of the staple may be connected to a stapler for delivering and crimping the staple and then detaching from the staple to permit removal of the stapler from the deployed staple.

In accordance with the invention, each of the prong groups 28a, 28b may include a primary prong 34 and one or more secondary prongs 36. When the staple is formed to its functioning configuration (FIG. 4), the sharp distal tips 35 of the primary prongs 34 will be located closer to the longitudinal central axis 29 of the staple than the sharp tips 37 of the secondary prongs 36. The secondary prongs 36 are splayed radially outwardly from central axis 29 at an angle that is greater than that of the primary prongs. The secondary prongs 36 may be configured so that they do not extend distally as far as the primary prongs. In the illustrative embodiment, the primary prongs 34 are embraced by a pair of secondary prongs 36.

In use, in the event that a primary prong 34 misses its target tissue, staple 20 is more likely to achieve effective staple-tissue engagement because splayed secondary prongs 36 define a wider span than the primary prongs. In the illustrative embodiment wherein secondary prongs 36 do not extend distally as far as the primary prongs 34, continued advancement of the staple 20 after a primary prong 34 misses its target tissue is more likely to result in effective staple-tissue engagement when the staple has been advanced sufficiently so that the secondary prongs 36, which define a wider span than the primary group, can engage the tissue. Thus, the staple 20 provides at least two groups of prongs including the primary prongs and a set of back-up or secondary prongs adapted to project radially outward more than the primary prongs and, therefore, more likely to engage tissue. The number of primary prongs may be varied, as may be the number and arrangement of secondary prongs.

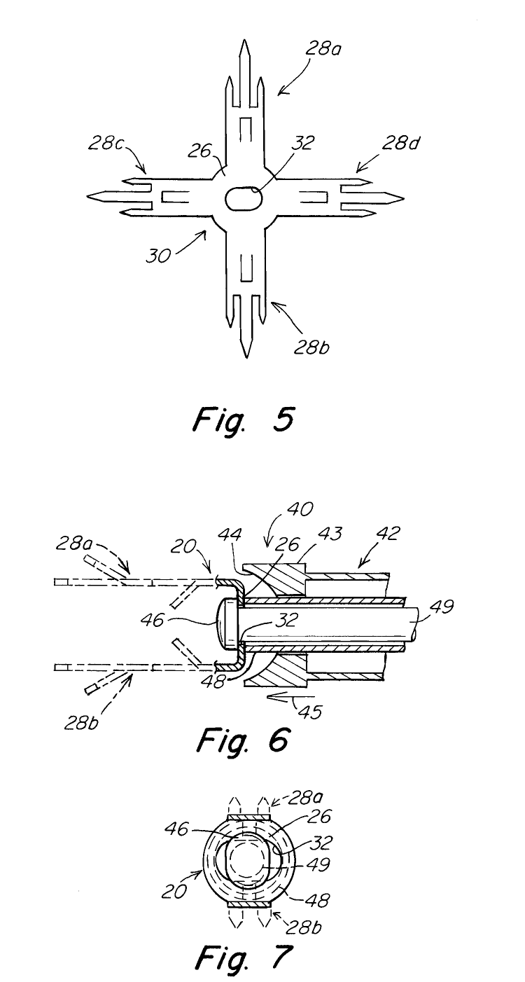

The staple also may be formed to include tissue stops 38. There may be stamped from the blank and oriented to project inwardly toward the axis 29 of the staple. The tissue stops 38 are employed to limit the extent to which the staple may be advanced through tissue. The tissue stops 38, preferably are located proximally of the most proximal secondary prong. FIG. 5 illustrates an embodiment of the staple 20 in which four such prong groups 28a, 28b, 28c and 28d, arranged in even spacing about the axis 29, are provided.

The illustrated device may be used, for example, with the type of stapler as described in U.S. Pat. No. 6,989,086, the disclosure of which is incorporated by reference, in its entirety. As shown in FIG. 6, the stapling elements 40 that engage and deploy the staple 20 are located at the distal end of a stapling device 42. The stapling arrangement includes a member 43 having a crimping surface 44 and a flange 46. The flange 46 is shaped to match the configuration of the non-circular aperture 32 in the crown 30 of the staple. In use, the staple is aligned with and is inserted over the flange 46 so that it abuts a shaft 48 adjacent the flange 46. The staple then is rotated 90 degrees to lock the staple 20 between the flange 46 and shaft (See FIG. 7). The flange 46 is connected to a rod 49 that passes through the stapler to the proximal end. The rod can be controlled to rotate the flange 46 into alignment with the aperture 32 when it is desired to release the staple after the staple has been deployed. During staple deployment, after the prongs have engaged tissue, the staple is crimped by member 43 having a crimping surface 44 that is advanced distally as suggested by the arrow 45. The crimping surface 44 may be parabolic to cause further deformation of the staple to crimp the prongs together.

It should be understood that the foregoing description of the invention is intended merely to be illustrative and that other embodiments, modifications and equivalents may be apparent to those skilled in the art while remaining within the scope of the invention.

Having described the invention, we claim:

1. An arteriotomy staple having proximal and distal ends with a crown at the proximal end and a plurality of prongs extending distally from the crown, the staple having a central axis and the prongs being disposed about the central axis, the staple comprising:
the prongs being arranged in groups, each group comprising at least one primary and at least one secondary prong, the distal tips of the secondary prongs being spaced radially from the central axis greater than the radial spacing of the distal tips of the primary prongs from the axis.

2. An arteriotomy staple as defined in claim 1 wherein the distal tips of the primary prongs extend distally beyond the digital tips of the secondary prongs.

3. An arteriotomy staple as defined in claim 1 wherein each group of prongs includes a central primary prong and a pair of secondary prongs embracing their associated primary prong.

4. An arteriotomy staple as defined in claim 1 further comprising means formed in the crown for enabling releasable connection of the staple to a stapler.

5. An arteriotomy staple as defined in claim 4 wherein the means comprises a noncircular aperture formed in the crown of the staple.

6. An arteriotomy staple as defined in claim 1 wherein the secondary prongs extend along a direction that makes an angle with the central axis that is greater than the angle between their associated primary prongs and the central axis.

7. An arteriotomy staple as defined in claim 1 wherein there are two diametrically opposed prong groups.

8. An arteriotomy staple as defined in claim 1 wherein there are four diametrically opposed prong groups.

9. An arteriotomy staple as defined in claim 1 wherein the prong groups are arranged symmetrically about the central axis.
